(19) United States
(12) Patent Application Publication
(10) Pub. No.: US 2008/0306381 A1
(43) Pub. Date: Dec. 11, 2008

(54) Method for optimizing the time sequence of examinations with the aid of a tomography system having a control unit by using contrast agents, a tomography system and contrast agent injector

(76) Inventors: Ute Feuerlein, Erlangen (DE); Gabriel Haras, Mucke (DE); Harald Nutzel, Forchheim (DE)

Correspondence Address: HARNESS, DICKEY & PIERCE, P.L.C., P.O.BOX 8910, RESTON, VA, 20195, US

(21) Appl. No.: 12/153,748

(22) Filed: May 23, 2008

(30) Foreign Application Priority Data
May 25, 2007 (DE) 10 2007 024 453.5

(51) Int. Cl. A61B 5/05

(52) U.S. Cl. 600/425; 600/431

(57) ABSTRACT

A method, a tomography system and a contrast agent injector for optimizing the time sequence of contrast agent examinations are disclosed, an automatically controlled text announcement system being used for instructing the behavior of a patient before or during the examination, and it being possible for a multiplicity of different announcement texts of different announcement duration to be temporally triggered and announced by the text announcement system. In at least one embodiment, on the basis of the announcement duration of one announcement text to be announced during the examination, the temporal behavior of this text relative to the work sequence of the contrast agent injector and/or of the tomography system is calculated, and inserted in the timing of the contrast agent injector via an electronic connection between the tomography system and the contrast agent injector is one time pause that leads to a synchronization of the work sequence of the contrast agent injector with the announcement duration of the current text announcement in such a way that the text announcement ends at a predetermined triggering instant of the combined work sequence of the tomography system and contrast agent injector.

### PRIORITY STATEMENT

The present application hereby claims priority under 35 U.S.C. §119 on German patent application number DE 10 2007 024 453.5 filed May 25, 2007, the entire contents of which is hereby incorporated herein by reference.

### FIELD

Embodiments of the invention generally relate to a method for optimizing the time sequence of examinations. For example, they may relate to a method for optimizing the time sequence of examinations with the aid of a tomography system having a control unit by using contrast agents, in which there are carried out the method steps of setting a prescribed time-controlled work sequence for the operation of an electronically controlled contrast agent injector, the contrast agent injector being connected in control terms to the tomography system at least in such a way that a scanning procedure can be started upon actuation of a starting apparatus (for example a start key, a start switch or a start button) at the contrast agent injector and of setting a prescribed time controlled work sequence for the operation of the tomography system, an automatically controlled text announcement system being used for instructing the behavior of a patient before or during the examination, and it being possible for a multiplicity of different announcement texts of different announcement duration to be temporally triggered and announced by the text announcement system, the temporal work sequences of a contrast agent injector and tomography system being interlinked.

Furthermore, embodiments of the invention also generally relate to a tomography system having a control and arithmetic unit, and to a contrast agent injector, there being in their memories program code for executing predefined methods.

Furthermore, embodiments of the invention also generally relate to a contrast agent injector for use in conjunction with a tomography system, the tomography system and the contrast agent injector each having a control and arithmetic unit with a memory in which there is program code for executing predefined methods.

### BACKGROUND

Tomography systems in particular NMR systems or CT systems, are generally known in conjunction with automatically operating contrast agent injectors that are integrated in the control of the tomography system. On the basis of safety regulations, the contrast agent injector is regarded here as an independent device that has in addition to the timing protocol of the scanner a dedicated timing protocol that it drives independently. However, on the basis of appropriate electronic connections the two systems are capable of executing start commands for the entire unit composed of tomography system and contrast agent injector. The examination can therefore be started from both systems.

Moreover, a malfunction of one of the systems is reported immediately to the other system, and so the two systems can carry out a functional breakdown independently of one another. Nevertheless, with the exception of the start the control of the time sequences of the two systems is performed independently of one another. This gives rise to problems with reference to a flexible timing of the two systems. If, for example, automatic text announcements in different languages are used in the tomography system, in terms of time, text announcements of identical significance have a substantially different announcement duration. If the timing is programmed in the contrast agent injector, it has so far been necessary to reprogram when changing from one language to the next. However, this is attended by substantial time outlay and possibilities of errors.

### SUMMARY

In at least one embodiment of the invention, a method, a combination of a tomography system with a contrast agent injector, and/or a contrast agent injector for a tomography system are disclosed, there in each case being the possibility of more flexibly influencing a preprogrammed timing in the contrast agent injector.

In at least one embodiment, the inventors have realized the following:

There are two main problems in the case of CT examinations with the aid of contrast agent, specifically the temporal coordination of CT scan and contrast agent injection, on the one hand, and the occurrence of movement artifacts, on the other hand. In order to achieve a temporal coordination between scan and injection, it is currently normal to apply one of the following methods.

Observation of a test bolus injection: in this method, before a diagnostic scan the inundation of a small quantity of contrast agent in a blood vessel is observed by repeated control scans. The time profile of the diagnostic examination is optimized by manual evaluation of this test examination.

Bolus tracking: this is an automated method in the case of which directly before a diagnostic scan the inundation of contrast agent in a blood vessel is measured by the tomography system by means of control scans, and the diagnostic scan is automatically started upon the overshooting of a threshold value.

The temporal coordination is supported by a technical coupling of the contrast agent injector and, for example, a CT scanner. In this case, the start command for the examination acts in the same way on the two devices and can also be given by two devices. In order to ensure that the beginning of the injection lies ahead of the scan in time, it is possible to define a scanning delay time. This is necessary because the contrast agent requires a circulation time in order to flow from the injection site to the organ, and to reach the desired concentration in the organ.

In order to avoid movement artifacts, it is very important that the patient hold his breath during the examination. For this reason, the patient is usually given a breathing command before the examination, for example “breathe in—breathe out—breathe in—cease breathing”. The breathing command can be performed in an automated fashion by a memory via a speech output.

The time duration of the automatic monitoring phase during bolus tracking—that is to say, the observation phase up to when a desired contrast agent concentration is reached—usually lasts 4 seconds, but can last substantially longer depending on the patient's circulation situation and the body region being examined. As a rule, a clinically sensible breathing command for this type of investigation lasts longer than 8 seconds.

If the aim is to operate with a conventional breathing command, for technical reasons the scanning delay (=scanning delay time)—the time between reaching the contrast agent concentration and start of the diagnostic scan—must be at least as long as the selected breathing command. The difference between a requisite scanning delay (for example 4 seconds) and a minimum possible scanning delay (for example 8 seconds) leads either to a belated starting instant of the diagnostic scan or to the fact that the breathing command has not yet ended at the starting instant of the scan. If this problem arises, there is generally a poor possibility of evaluating the examination since, for example, the arterial phase cannot be detected, or there is an examination affected by respiration. For these reasons, optimizing the work flow by using automatic breathing commands in conjunction with automatic bolus tracking has so far been renounced in practice.

According to at least one embodiment of the invention, this problem can be improved upon or even solved by virtue of the fact that flexible influencing of the timing of the contrast agent injector by the tomography system can be implemented in a simple way without having to intervene in the basic structure of a contrast agent injector and its program flow. To this end, a possibility is created for inserting pauses of any desired duration at one or more points in the timing as a function of the announcement texts used or of the different announcement duration thereof.

It is possible to this end, for example, to transmit a control signal, where appropriate with a data record, to a contrast agent injector when use is made of automatic bolus tracking before the monitoring phase, but after knowledge of the announcement duration to be used for the announcement text or the announcement texts, as a result of which an automatically calculated “injection pause” is inserted into the contrast agent protocol of the injector at a desired point in the timing.

Here, the injection pause is calculated from the difference between the length of the breathing command, or of the respectively selected breathing commands, and the scanning delay set.

The temporal variability of the monitoring phase, for example in the case of a reduced heart minute volume, or the examination of peripheral body regions, can cause a long additional breath holding phase. Since the patients are generally unwell, they can, however, hold their breath only over a relatively short time period. There is thus once again the risk of obtaining an examination result that is affected by respiration and therefore has a poor possibility of evaluation.

In an advantageous design, the length of the “injection pause” can respectively be adapted to the individual examination situation automatically via a configuration or manually by the user.

The expected length of the monitoring phase can be estimated on the basis of biometric and physiological data. If appropriate data are documented in a first examination, for example an ECG and the circulation time determined by bolus tracking, it is possible during a second examination to infer the circulation time currently to be expected, doing so by putting it into relationship with the current heart rate. The “injection pause” can be automatically adapted in an appropriate way.

The data can be held ready in a system database or be managed via other storage methods that, for example, are offered by a hospital information system (HIS).

The method can be applied with the aid of the most varied biometric and physiological data. A further example could be the heart minute volume, already determined by other examinations, of the patient in relation to the current ECG.

Further examples from which rules can be derived are:
spacing between the monitoring plane and the starting position of the diagnostic scan (scanning range planning data);
data of a test bolus examination;
patient weight, patient size;
flow properties of the contrast agent, for example as a function of temperature or concentration; and
distance of the position of the injection site from the observed organ.

In accordance with these basic ideas, at least one embodiment of the inventors propose that the method known per se be used to optimize the time sequence of examinations with the aid of a tomography system having a control unit by using contrast agents and comprising the following method steps:
setting a prescribed time controlled work sequence for the operation of an electronically controlled contrast agent injector, the contrast agent injector being connected in control terms to the tomography system at least in such a way that a scanning procedure can be started upon actuation of a starting apparatus (for example a start key, a start switch or a start button) at the contrast agent injector or at the tomography system, and
setting a prescribed time controlled work sequence for the operation of the tomography system, an automatically controlled text announcement system being used for instructing the behavior of a patient before or during the examination, and it being possible for a multiplicity of different announcement texts of different announcement duration to be temporally triggered and announced by the text announcement system, the temporal work sequences of a contrast agent injector and tomography system being interlinked, in the following way:
on the basis of the known announcement duration of at least one announcement text currently to be announced during the planned examination, the temporal behavior of this text relative to the work sequence of the contrast agent injector and/or of the tomography system is calculated, and
inserted at least in the timing of the contrast agent injector via an electronic connection between the tomography system and the contrast agent injector is at least one time pause that leads to a synchronization of the work sequence of the contrast agent injector with the announcement duration of the current text announcement in such a way that the text announcement ends at a predetermined triggering instant of the combined work sequence of the tomography system and contrast agent injector.

It is rendered possible by means of such a measure to adapt the method sequence temporally to the facts of different announcement texts, in particular their announcement duration, it being possible to avoid intervening in the actual program flow of the contrast agent injector.

It is proposed in accordance with one variant of the method that when calculating the temporal behavior of the announcement text relative to the work sequence of the contrast agent injector and/or of the tomography system, the announcement duration of a starting text announced at the beginning of the examination is compared with the prescribed time interval between the beginning of the injection and the start of a monitoring phase, and in the event of a longer lasting announcement duration there is inserted between the receipt of the start command and the beginning of the injection a pause that corresponds to the difference between the announcement duration and the time interval between the beginning of the injection and the start of the monitoring phase.

It is also possible that with reference to an announcement of a text between a first and a second triggering event in the combined work sequence of the tomography system and contrast agent injector, the time between the two triggering events is compared with the announcement duration of the announcement text, and for a longer lasting announcement text an additional pause, corresponding to the difference, between a first and second triggering event is inserted at least in the work sequence of the tomography system and/or of the contrast agent injector.

Furthermore, the insertion of the pauses in the programmed timing can take place at least partially before the beginning of the examination and after selection of the at least one announcement text.

The insertion of the pauses in the programmed timing can also take place at least partially after the beginning of the examination on the basis of triggering events occurring during the examination. By way of example, it is possible in this case to select the reaching of an HU value limit during a monitoring approach as first triggering event, and the start of the examination scan as second triggering event.

If it is desired with reference to the time sequences in the body of a patient after a bolus injection to rely not only on estimated values or mean values or limiting values from earlier statistical examinations, there is the possibility in accordance with an advantageous refinement of the proposed method that before the beginning of the examination sequence the temporal behavior of a test bolus injection is also examined, and the timings are matched by inserting pauses in the prescribed work sequence of the tomography system and/or of the contrast agent injector.

Use is mostly made of announcement texts that constitute breathing commands, such as: “Breathe in . . . breathe out . . . hold the breath . . . breathe again”, or the like.

Such texts can either be directly input or played back, but it is also possible to assemble the entire text from a plurality of stored partial texts. Such an announcement text can, for example, be recorded before the examination, or it can be generated artificially from written text.

It is chiefly X-ray computed tomography systems or NMR tomography systems (=nuclear magnetic resonance imaging systems) that are provided as tomography systems within the meaning of at least one embodiment of the invention, but at least one embodiment of the invention can also be applied to other known types of tomography systems, and also for therapeutic irradiation systems in which there is a need to give verbal commands in the work sequence of an examination, and where, for example, use is made of different languages.

Also within the scope of at least one embodiment of the invention is a tomography system having a control and arithmetic unit and a contrast agent injector, each with a memory in which there is program code for executing predefined methods, there also being stored program code for carrying out at least one embodiment of the method when the system is operating.

Likewise within the scope of at least one embodiment of the invention is a contrast agent injector for use in conjunction with a tomography system, the tomography system and the contrast agent injector each having a control and arithmetic unit with a memory in which there is program code for executing predefined methods, there also being stored program code for carrying out at least one embodiment of the method when the system is operating.

At least one embodiment of the proposed method can now be used to select the length of a breathing command independently of the scanning delay even when use is made of automatic bolus tracking.

An advantage of at least one embodiment resides in the fact that contrast agent examinations become simpler and quicker for the user and safer for the patient. By comparison with the prior art, at least one of the following advantages are further achieved by way of at least one embodiment of the abovedescribed invention:
patients who speak foreign languages can be examined optimally, since the breathing commands are stored in various languages;
improved contrast timing;
reduction of movement artifacts;
avoidance of maloperations;
improvement of recording quality;
improvement of the reproducibility of the recordings.

### BRIEF DESCRIPTION OF THE DRAWINGS

The invention is explained in more detail below with the aid of preferred example embodiments with the aid of the figures, only the features required to understand the invention being represented. Use is made here of the following reference symbols: 1: CT system; 2: first X-ray tube; 3: first detector; 4: second X-ray tube; 5: second detector; 6: gantry housing; 7: patient; 8: patient couch; 9: system axis; 10: control and arithmetic unit; 10.1: start button; 11: contrast agent injector; 11.1: start button; 101: monitoring delay; 102: monitoring phase; 103: scanning delay; 104: scanning duration; 110: start command; 111: start of the monitoring; 112: reaching of a triggering level; 113: scanning start; 114: scanning end; 200: start command; 201: monitoring delay; 202: monitoring phase; 203: scanning delay; 204: scanning duration; 210: beginning of the injection of the contrast agent; 211: start of the monitoring; 212: reaching of a triggering level; 213: scanning start; 214: scanning end; D₁, D₂, D₃: duration of the announcement text; Prg₁-Prgₙ: computer program; Prgₓ: computer program (Prgₓ⊂Prg₁-Prgₙ); P₁: pause; t: time.

In detail:

FIG. 1 shows the timing of a scanning examination without a previous test bolus examination, with and without an inserted pause;

FIG. 2 shows the timing of a scanning examination with a previous test bolus examination, with and without an inserted pause; and

FIG. 3 shows an X-ray computed tomography system having a contrast agent injector.

### DETAILED DESCRIPTION OF THE EXAMPLE EMBODIMENTS

Various example embodiments will now be described more fully with reference to the accompanying drawings in which only some example embodiments are shown. Specific structural and functional details disclosed herein are merely representative for purposes of describing example embodiments. The present invention, however, may be embodied in many alternate forms and should not be construed as limited to only the example embodiments set forth herein.

Accordingly, while example embodiments of the invention are capable of various modifications and alternative forms, embodiments thereof are shown by way of example in the drawings and will herein be described in detail. It should be understood, however, that there is no intent to limit example embodiments of the present invention to the particular forms disclosed. On the contrary, example embodiments are to cover all modifications, equivalents, and alternatives falling within the scope of the invention. Like numbers refer to like elements throughout the description of the figures.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items.

It will be understood that when an element is referred to as being “connected,” or “coupled,” to another element, it can be directly connected or coupled to the other element or intervening elements may be present. In contrast, when an element is referred to as being “directly connected,” or “directly coupled,” to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Spatially relative terms, such as “beneath”, “below”, “lower”, “above”, “upper”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, term such as “below” can encompass both an orientation of above and below. The device may be otherwise oriented (rotated 90 degrees or at other orientations) and the spatially relative descriptors used herein are interpreted accordingly.

Although the terms first, second, etc. may be used herein to describe various elements, components, regions, layers and/or sections, it should be understood that these elements, components, regions, layers and/or sections should not be limited by these terms. These terms are used only to distinguish one element, component, region, layer, or section from another region, layer, or section. Thus, a first element, component, region, layer, or section discussed below could be termed a second element, component, region, layer, or section without departing from the teachings of the present invention.

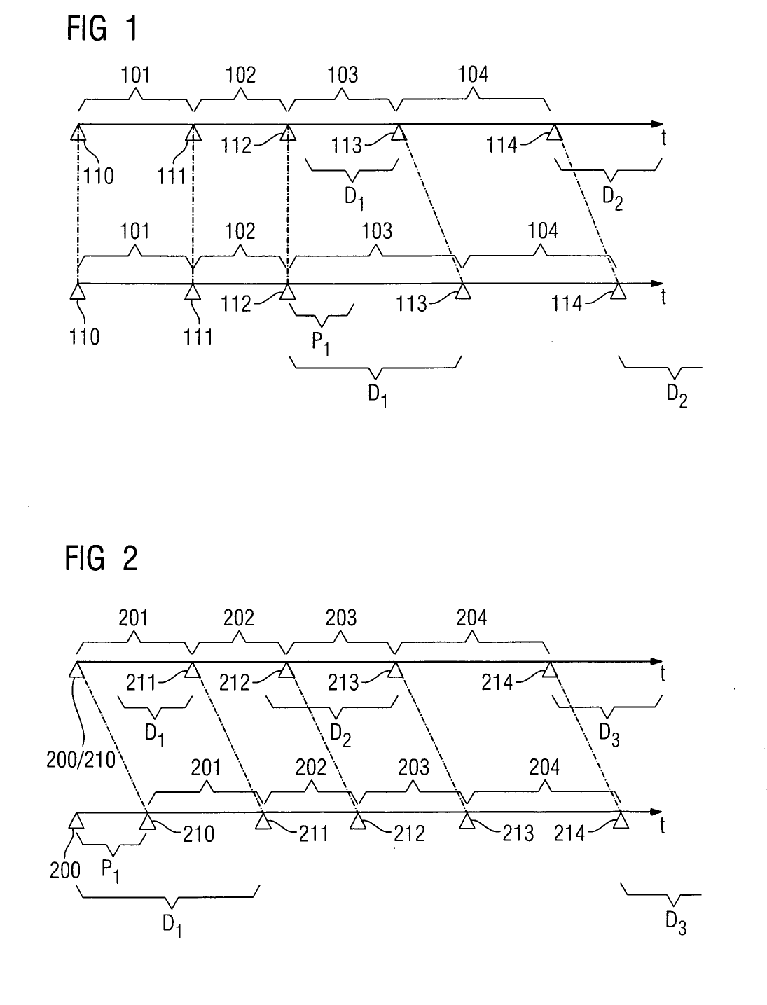

FIG. 1 shows the timing of a scanning examination without a previous test bolus examination, for the purpose of determining the reaction time of the injection of a contrast agent up to the contrast enhancement in the observed organ, with and without an inventively inserted pause in the profile protocol. The time t is plotted on the time axis illustrated at the top, the time intervals 101 to 104 with 101 the monitoring delay—that is to say, the delay from the starting instant 110 (=instant of the execution of the start command) up to the beginning of the monitoring 111—, 102 the duration of the monitoring up to the reaching of a triggering level 112—that is to say, of a predetermined contrast range or HU value on the basis of sufficient contrast agent concentration—, 103 the scanning delay—that is to say, the scanning delay between the triggering level 112 up to the scanning start 113, and 104 the scanning duration from the scanning start 113 up to the scanning end 114. In the case of this time profile, there is no pause inserted in the region of the scanning delay, since in the comparison of the announcement text used the duration D₁ of the announcement text that is to be accommodated in the scanning delay with announcement end at the scanning start is smaller than the scanning delay 103 that is set in any case.

The time trace shown directly therebelow shows the situation, identical per se, of the timing of a scanning examination without a previous test bolus examination, although because of the selected announcement text, which has a longer announcement duration D₁ than in the example above, this announcement duration is also longer than the scanning delay 103. Since the time duration of the monitoring phase 102 is, however, not known with certainty, it is also not possible to make use for the beginning of the excessively long announcement text simply of an instant advanced into the monitoring phase 102. Thus, according to an embodiment of the invention once the excessively long announcement duration D₁ relative to the set scanning delay 103 is known, a pause P₁ is subsequently inserted at the end of the monitoring phase 102 in order to extend the scanning delay 103. There is thus sufficient time between the reaching of the triggering level 112 and the start of the scan 113 in order to output the announcement text during this additionally extended phase 103.

The dashed and dotted lines between two similar time traces illustrate the time shifting of the instants 113 and 114 relative to one another, while the instants 110, 111 and 112 are preserved. In the case of both timings, a second announcement text with the announcement duration D₂ is added to the end of the diagnostic scan at 114′ at the end of the examination.

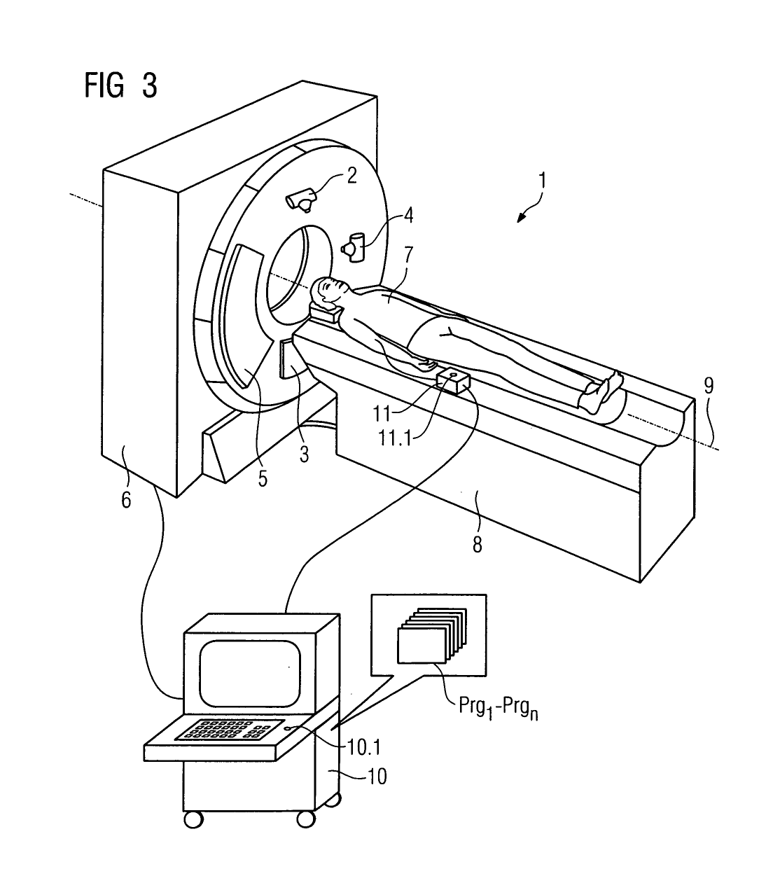

Another variant of an embodiment of the inventive method is illustrated in FIG. 2. Here, the timing of a scanning examination after a test bolus examination carried out previously is shown with and without an inserted pause P₁. In a way similar to FIG. 1, the timing begins with the start command 200, which also corresponds to the beginning of the injection of the contrast agent 210. There follows the phase of the monitoring delay 201 up to the starting instant 211 of the monitoring phase 202. However, this example involves only a control monitoring, since the temporal rise in the contrast agent in the observed organ is already known owing to the prior investigation with test bolus administration. There follows subsequently a scanning delay 203 between the reaching of the triggering level 212 and the start of the diagnostic scan 213 in the course of which, for example, the correct positioning of the patient for the diagnostic scan can be carried out such that a subsequent spiral scan begins at the correct z position of the patient. The diagnostic scan 204 up to the end of the scan 214 follows after the start 213. In this example, the duration D₃ of a first announcement text is shorter than the duration of the monitoring delay 201, and so there is no need to influence the timing. Since the instant at which the triggering level is reached is known quite accurately owing to the preceding test bolus examination, the second announcement text with the announcement duration D₂ can also already begin during the monitoring phase 202, and so there is no need to change the scanning delay 203 despite the time overshooting of the second announcement text.

The time profile of the lower time trace, in the case of which the first announcement text has a longer announcement duration D₃ than the duration of the monitoring delay 201 programmed in, is of different appearance. Consequently, a pause P₁ is subsequently inserted at the starting instant 200 (=pressing of the start button) whereupon the beginning of the injection 210 is delayed by the contrast agent injector such that the first announcement text with the announcement duration D₃ fits between the starting instant 200 and the start of the monitoring phase 211. The subsequent timing corresponds again to the previously described timing of the upper time trace from this figure. In the case of both frequencies, a second announcement text with the announcement duration D₂ occurs upon ending of the diagnostic scan 204, but it is no longer relevant to the timing.

Thus, a pause is inserted in accordance with an embodiment of the invention in a fashion dependent on the prescribed timing of the contrast agent injector at respectively required points so as to enable a simple and flexible adaptation to different time requirements of a tomography examination with administration of contrast agent without intervention in the control of the contrast agent injector and without program change.

An embodiment of the inventive method described above can be integrated in different embodiments, for example in an X-ray CT system and, in particular, in a contrast agent injector connected thereto. Such a CT system 1 is illustrated by way of example in FIG. 3. This CT system 1 has a gantry housing 6 in which there are arranged with an angular offset two X-ray tubes 2 and 4 with opposite detector systems 3 and 5 that rotate about a system axis 9 in order to scan the patient 7, while the patient 7 is displaced along the system axis 9 through the measurement area of the CT system by the controllable patient couch. Control, reconstruction and carrying out an embodiment of the inventive method are served by a control and arithmetic unit 10 that includes in its memory computer programs Prg₁-Prgₙ that carry out the control and reconstruction during operation. At least one program Prgₓ that carries out an embodiment of an inventive method is also part of these programs. Also connected to the control and arithmetic unit is a contrast agent injector 11 with which it is possible to apply contrast agent with the aid of a programmed timing, it being possible in accordance with the abovedescribed method to insert pauses in a desired way at predetermined points of the timing by driving the contrast agent injector 11 by means of the control and arithmetic unit 10 of the CT system.

It goes without saying that the abovementioned features of the invention can be used not only in the combination respectively specified, but also in other combinations or on their own without departing from the scope of the invention.

Further, elements and/or features of different example embodiments may be combined with each other and/or substituted for each other within the scope of this disclosure and appended claims.

Still further, any one of the above-described and other example features of the present invention may be embodied in the form of an apparatus, method, system, computer program and computer program product. For example, of the aforementioned methods may be embodied in the form of a system or device, including, but not limited to, any of the structure for performing the methodology illustrated in the drawings.

Even further, any of the aforementioned methods may be embodied in the form of a program. The program may be stored on a computer readable media and is adapted to perform any one of the aforementioned methods when run on a computer device (a device including a processor). Thus, the storage medium or computer readable medium, is adapted to store information and is adapted to interact with a data processing facility or computer device to perform the method of any of the above mentioned embodiments.

The storage medium may be a built-in medium installed inside a computer device main body or a removable medium arranged so that it can be separated from the computer device main body. Examples of the built-in medium include, but are not limited to, rewriteable non-volatile memories, such as ROMs and flash memories, and hard disks. Examples of the removable medium include, but are not limited to, optical storage media such as CD-ROMs and DVDs; magneto-optical storage media, such as MOs; magnetism storage media, including but not limited to floppy disks (trademark), cassette tapes, and removable hard disks; media with a built-in rewriteable non-volatile memory, including but not limited to memory cards; and media with a built-in ROM, including but not limited to ROM cassettes; etc. Furthermore, various information regarding stored images, for example, property information, may be stored in any other form, or it may be provided in other ways.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed:

1. A method for optimizing the time sequence of examinations with the aid of a tomography system having a control unit by using contrast agents, the method comprising:
setting a prescribed time controlled work sequence for operation of an electronically controlled contrast agent injector, the contrast agent injector being connected in control terms to the tomography system at least in such a way that a scanning procedure is started upon actuation of a starting apparatus at least one of at the contrast agent injector and at the tomography system;
setting a prescribed time controlled work sequence for the operation of the tomography system, an automatically controlled text announcement system being used for instructing the behavior of a patient before or during the examination, and a multiplicity of different announcement texts of different announcement duration being temporally triggered and announced by the text announcement system, the temporal work sequences of a contrast agent injector and tomography system being interlinked;
calculating, on the basis of the known announcement duration of at least one announcement text currently to be announced during the planned examination, the temporal behavior of this text relative to the work sequence of at least one of the contrast agent injector and the tomography system; and
inserting, at least in the timing of the contrast agent injector via an electronic connection between the tomography system and the contrast agent injector, at least one time pause that leads to a synchronization of the work sequence of the contrast agent injector with the announcement duration of the current text announcement in such a way that the text announcement ends at a predetermined triggering instant of the combined work sequence of the tomography system and contrast agent injector.

2. The method as claimed in claim 1, wherein, when calculating the temporal behavior of the announcement text relative to the work sequence of at least one of the contrast agent injector and the tomography system, the announcement duration of a starting text announced at the beginning of the examination is compared with the prescribed time interval between the beginning of the injection and the start of a monitoring phase, and in the event of a longer lasting announcement duration, there is inserted, between the receipt of the start command and the beginning of the injection, a pause that corresponds to the difference between the announcement duration and the time interval between the beginning of the injection and the start of the monitoring phase.

3. The method as claimed in claim 1, wherein, for an announcement of a text between a first and a second triggering event in the combined work sequence of the tomography system and contrast agent injector, the time between the two triggering events is compared with the announcement duration of the announcement text, and for a longer lasting announcement text an additional pause, corresponding to the difference, between a first and second triggering event is inserted at least in the work sequence of at least one of the tomography system and the contrast agent injector.

4. The method as claimed in claim 1, wherein the insertion of the pauses in the programmed timing takes place at least partially before the beginning of the examination and after selection of the at least one announcement text.

5. The method as claimed in claim 1, wherein the insertion of the pauses in the programmed timing takes place at least partially after the beginning of the examination on the basis of triggering events occurring during the examination.

6. The method as claimed in claim 5, wherein the first triggering event is the reaching of an HU value limit during a monitoring approach, and the second triggering event is the start of the examination scan.

7. The method as claimed in claim 1, wherein, before the beginning of the examination sequence, the temporal behavior of a test bolus injection is examined, and the timings are matched by inserting pauses in the prescribed work sequence of at least one of the tomography system and the contrast agent injector.

8. The method as claimed in claim 1, wherein at least one announcement text gives at least one breathing command.

9. The method as claimed in claim 1, wherein at least one announcement text is assembled from a number of announcement texts.

10. The method as claimed in claim 1, wherein at least one announcement text is recorded before the examination.

11. The method as claimed in claim 1, wherein at least one announcement text is generated artificially from written text.

12. The method as claimed in claim 1, wherein an X-ray computed tomography system is used.

13. The method as claimed in claim 1, wherein an NMR (nuclear magnetic resonance imaging) tomography system is used.

14. A tomography system, comprising:
a control and arithmetic unit; and
a contrast agent injector, each of the control and arithmetic unit and contrast agent injector including a memory including program code for executing defined methods, at least one of the control and arithmetic unit and contrast agent injector memories including program code for carrying out the method of claim 1 when the tomography system is operating.

15. A contrast agent injector for use in conjunction with a tomography system, the tomography system and the contrast agent injector each including a control and arithmetic unit with a memory including program code for executing defined methods, at least one of the tomography system and the contrast agent injector control and arithmetic units including a memory program code for carrying out the method of claim 1 when the system is operating.

16. The method as claimed in claim 2, wherein, for an announcement of a text between a first and a second triggering event in the combined work sequence of the tomography system and contrast agent injector, the time between the two triggering events is compared with the announcement duration of the announcement text, and for a longer lasting announcement text an additional pause, corresponding to the difference, between a first and second triggering event is inserted at least in the work sequence of at least one of the tomography system and the contrast agent injector.

17. A tomography system, comprising:
a contrast agent injector;
means for setting a prescribed time controlled work sequence for operation of an electronically controlled contrast agent injector, the contrast agent injector being connected in control terms to the tomography system at least in such a way that a scanning procedure is started upon actuation of a starting apparatus at least one of at the contrast agent injector and at the tomography system;
means for setting a prescribed time controlled work sequence for the operation of the tomography system, an automatically controlled text announcement system being used for instructing the behavior of a patient before or during the examination, and a multiplicity of different announcement texts of different announcement duration being temporally triggered and announced by the text announcement system, the temporal work sequences of a contrast agent injector and tomography system being interlinked;
means for calculating, on the basis of the known announcement duration of at least one announcement text currently to be announced during the planned examination, the temporal behavior of this text relative to the work sequence of at least one of the contrast agent injector and the tomography system; and
means for inserting, at least in the timing of the contrast agent injector via an electronic connection between the tomography system and the contrast agent injector, at least one time pause that leads to a synchronization of the work sequence of the contrast agent injector with the announcement duration of the current text announcement in such a way that the text announcement ends at a predetermined triggering instant of the combined work sequence of the tomography system and contrast agent injector.

18. A contrast agent injector for use in conjunction with a tomography system, at least one of the tomography system and the contrast agent injector including,
means for setting a prescribed time controlled work sequence for operation of an electronically controlled contrast agent injector, the contrast agent injector being connected in control terms to the tomography system at least in such a way that a scanning procedure is started upon actuation of a starting apparatus at least one of at the contrast agent injector and at the tomography system;
means for setting a prescribed time controlled work sequence for the operation of the tomography system, an automatically controlled text announcement system being used for instructing the behavior of a patient before or during the examination, and a multiplicity of different announcement texts of different announcement duration being temporally triggered and announced by the text announcement system, the temporal work sequences of a contrast agent injector and tomography system being interlinked;
means for calculating, on the basis of the known announcement duration of at least one announcement text currently to be announced during the planned examination, the temporal behavior of this text relative to the work sequence of at least one of the contrast agent injector and the tomography system; and
means for inserting, at least in the timing of the contrast agent injector via an electronic connection between the tomography system and the contrast agent injector, at least one time pause that leads to a synchronization of the work sequence of the contrast agent injector with the announcement duration of the current text announcement in such a way that the text announcement ends at a predetermined triggering instant of the combined work sequence of the tomography system and contrast agent injector.

19. A computer readable medium including program segments for, when executed on a computer device, causing the computer device to implement the method of claim 1.
